(19) United States
(12) Patent Application Publication
(10) Pub. No.: US 2026/0065468 A1
(43) Pub. Date: Mar. 5, 2026

(54) METHOD AND SYSTEM FOR GENERATING MEDICAL REPORT BASED ON ACTIVATION PROMPTS

(71) Applicant: L&T TECHNOLOGY SERVICES LIMITED, Chennai (IN)

(72) Inventors: AJITH KOLAR JAYASHANKARA, Mysuru (IN); NETHRAVATHI AGATHAGOWDANAHALLI MAHADEVAIAH, Chamrajnagar (IN)

(21) Appl. No.: 19/019,590

(22) Filed: Jan. 14, 2025

(30) Foreign Application Priority Data
Sep. 5, 2024 (IN) 202441067396

(51) Int. Cl. G06T 7/00; G06T 7/11; G16H 15/00

(52) U.S. Cl. CPC G06T 7/0012; G06T 7/11; G16H 15/00; G06T 2207/30096

(57) ABSTRACT

A method and system for generating medical report is provided. A set of images comprising a body part are displayed on a display device. Further, a voice input is received from a user via an input device. A set of segmented images from each of the set of images are determined using a segmentation model based on detection of a first activation prompt from a pre-defined set of activation prompts in the voice input. A volumetric information of the anomaly in the body part from each of the set of segmented images is determined. Further, text data from the voice input is determined using a speech-to-text model based on detection of a second activation prompt. A medical report is generated based on the text data and the volumetric information.

### TECHNICAL FIELD

This disclosure relates generally to speech to text generation, and more particularly to a method and system for generating medical reports using speech to text generation.

### BACKGROUND

Diagnosis of anomalies such as tumours, cancer, ulcers etc. in the human body is performed through medical imaging such as x-rays, CT-scans, MRI images, etc. Diagnosis through medical imaging is essential for medical report generation to perform effective treatment strategies. Diagnosis is captured in medical reports that provides valuable insights into the anomaly and enables doctors in planning appropriate treatments based on the diagnosis.

Diagnosis is performed by medical practitioners such as a radiologist by closely analysing the medical images. Conventional methods for medical reporting are filled with challenges due to manual practices. Also, radiologists rely on manual assessment of anomaly such as tumours using the medical imaging studies, followed by a detailed analysis of tumour properties. Also, radiologists manually documented their findings, often using free-text reporting, which lack standardization and may be prone to errors. In some case, the radiologist may verbally dictate their diagnosis and tabulation of the analysis to prepare reports is often performed by an assistant or a trainee. Such manual approach could lead to incomplete reports or variations in the interpretation of imaging results, ultimately affecting patient care. Also, the manual process is labour-intensive, requiring significant time and expertise and is prone to human error. The accuracy of these manual methods can vary based on the radiologist's experience and the complexity of the case, leading to potential inconsistencies in diagnosis. Therefore, there arises a requirement of advanced tools to assist radiologists in their analysis and report preparation.

### SUMMARY OF THE INVENTION

In an embodiment, a method of generating a medical report is disclosed. The method may include displaying, by a processor, a set of images including a body part on a display device. Further, the method may include receiving, by the processor, a voice input from a user via an input device. The method may further include determining, by the processor, a set of segmented images from each of the set of images by segmenting the body part, an anomaly in the body part and a background using a segmentation model. In an embodiment, the set of segmented images are determined based on detection of a first activation prompt from a pre-defined set of activation prompts in the voice input. The method may further include determining, by the processor, a volumetric information of the anomaly in the body part from each of the set of segmented images. In an embodiment, the volumetric information may include volume information of the anomaly and area information of the anomaly determined in each of the segmented images. The processor may further determine, text data from the voice input using a speech-to-text model based on the detection of a second activation prompt from the pre-defined set of activation prompts in the voice input. Further, the method may include generating, by the processor, the medical report based on the text data and the volumetric information.

In another embodiment, a system of generating a medical report is disclosed. The system may include a processor, a memory communicably coupled to the processor, wherein the memory may store processor-executable instructions, which when executed by the processor may cause the processor to display a set of images including a body part on a display device. Further, the processor may receive a voice input from a user via an input device. The processor may further determine a set of segmented images from each of the set of images by segmenting the body part, an anomaly in the body and a background using a segmentation model. In an embodiment, the set of segmented images are determined based on detection of a first activation prompt from a pre-defined set of activation prompts in the voice input. The processor may further determine a volumetric information of the anomaly in the body part from each of the set of segmented images. In an embodiment, the volumetric information may include volume information of the anomaly and area information of the anomaly determined in each of the set of segmented images. The processor may further determine text data from the voice input using a speech-to-text model based on detection of a second activation prompt from the pre-defined set of activation prompts in the voice input. Further, the processor may generate the medical report based on the text data and the volumetric information.

Various objects, features, aspects, and advantages of the inventive subject matter will become more apparent from the following detailed description of preferred embodiments, along with the accompanying drawing figures in which like numerals represent like components.

### BRIEF DESCRIPTION OF THE DRAWINGS

The accompanying drawings, which are incorporated in and constitute a part of this disclosure, illustrate exemplary embodiments and, together with the description, serve to explain the disclosed principles.

FIG. 1 illustrates a block diagram of an exemplary medical report generation system based on activation prompts, in accordance with some embodiments of the present disclosure.

FIG. 2 is a functional block diagram of a computing device, in accordance with some embodiments of the present disclosure.

FIG. 3A illustrates exemplary set of images of a body part, in accordance with some embodiments of the present disclosure.

FIG. 3B illustrates exemplary set of segmented images of the set of images of FIG. 3A, in accordance with some embodiments of the present disclosure.

FIG. 3C illustrates exemplary set of post-processed images of the exemplary set of segmented images of FIG. 3B, in accordance with some embodiments of the present disclosure.

FIG. 4 illustrates a flow diagram of a methodology of generating medical report, in accordance with some embodiments of the present disclosure.

### DETAILED DESCRIPTION OF THE DRAWINGS

Exemplary embodiments are described with reference to the accompanying drawings. Wherever convenient, the same reference numbers are used throughout the drawings to refer to the same or like parts. While examples and features of disclosed principles are described herein, modifications, adaptations, and other implementations are possible without departing from the scope of the disclosed embodiments. It is intended that the following detailed description be considered exemplary only, with the true scope being indicated by the following claims. Additional illustrative embodiments are listed.

Further, the phrases “in some embodiments”, “in accordance with some embodiments”, “in the embodiments shown”, “in other embodiments”, and the like mean a particular feature, structure, or characteristic following the phrase is included in at least one embodiment of the present disclosure and may be included in more than one embodiment. In addition, such phrases do not necessarily refer to the same embodiments or different embodiments. It is intended that the following detailed description be considered exemplary only, with the true scope and spirit being indicated by the following claims.

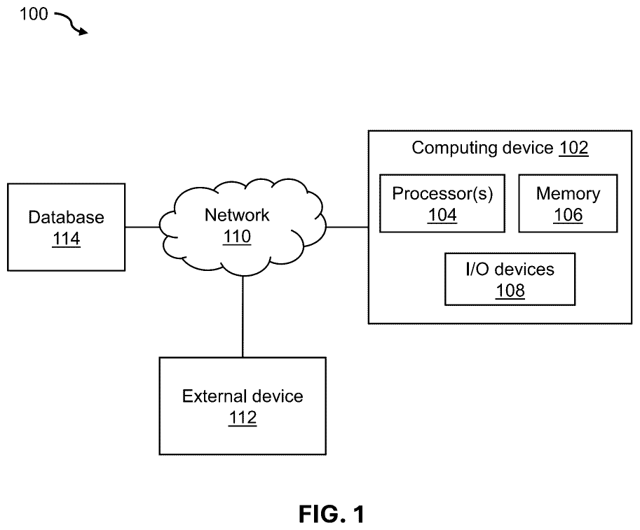

Referring now to FIG. 1, illustrates a block diagram of an exemplary medical report generation system 100 for generating a medical report, in accordance with an embodiment of the present disclosure. The medical report generation system 100 may include a computing device 102, an external device 112 and a database 114, communicably coupled to each other through a wired or a wireless communication network 110. The computing device 102 may include a processor 104, a memory 106 and an input/output (I/O) device 108. In an embodiment, examples of processor 104 may include, but are not limited to, an Intel® Itanium® or Itanium 2 processor(s), or AMD® Opteron® or Athlon MP® processor(s), Motorola® lines of processors, Nvidia®, FortiSOC™ system on a chip processors or other future processors. In an embodiment, the memory 106 may store instructions that, when executed by the processor 104, may cause the processor 104 to generate medical report as discussed in more detail below. In an embodiment, the memory 106 may be a non-volatile memory or a volatile memory. Examples of non-volatile memory may include but are not limited to, a flash memory, a Read Only Memory (ROM), a Programmable ROM (PROM), Erasable PROM (EPROM), and Electrically EPROM (EEPROM) memory. Further, examples of volatile memory may include but are not limited to, Dynamic Random Access Memory (DRAM), and Static Random-Access memory (SRAM).

In an embodiment, the I/O devices 108 may include a display device (not shown) and a mic (not shown). The I/O devices 108 may comprise of variety of interface(s), for example, interfaces for data input and output, and the like. The I/O devices 108 may facilitate inputting of instructions by a user communicating with the computing device 102. In an embodiment, the I/O devices 108 may be wirelessly connected to the computing device 102 through wireless network interfaces such as Bluetooth®, infrared, or any other wireless radio communication known in the art. In an embodiment, the I/O devices 108 may be connected to a communication pathway for one or more components of the computing device 102 to facilitate the transmission of inputted instructions and output results of data generated by various components such as, but not limited to, processor(s) 104 and memory 106.

In an embodiment, the database 114 may be enabled in a remote cloud server or a co-located server. In an embodiment, the database 114 and may include a database to store an application, medical imaging data, and other data necessary for the system 100 to generate medical report. In an embodiment, the database 114 may store data to be input to the computing device 102 or output generated by the computing device 102.

In an embodiment, the communication network 110 may be a wired or a wireless network or a combination thereof. The communication network 110 can be implemented as one of the different types of networks, such as, but not limited to, ethernet IP network, intranet, local area network (LAN), wide area network (WAN), the internet, Wi-Fi, LTE network, CDMA network, 5G and the like. Further, the communication network 110 can either be a dedicated network or a shared network. The shared network represents an association of the different types of networks that use a variety of protocols, for example, Hypertext Transfer Protocol (HTTP), Transmission Control Protocol/Internet Protocol (TCP/IP), Wireless Application Protocol (WAP), and the like, to communicate with one another. Further the communication network 110 can include a variety of network devices, including routers, bridges, servers, computing devices, storage devices, and the like.

In an embodiment, the computing device 102 and the external device 112 may be a computing system, including but not limited to, a smart phone, a laptop computer, a desktop computer, a notebook, a workstation, a portable computer, a personal digital assistant, a handheld, a scanner, or a mobile device. In an embodiment, the computing device 102 may be, but not limited to, in-built into the external device 112 or may be a standalone computing device.

In an embodiment, the computing device 102 may perform various processing for generating a medical report. By way of an example, the computing device 102 may display a set of images that may include a body part on a display device of the I/O devices 108. In an embodiment, the set of images may include, but not limited to, computed tomography (CT) scan images of body part of a patient captured using a CT scan machine. Further, the computing device 102 may receive a voice input from a user via the input device 108 such as a microphone. In an embodiment, the user may be a radiologist or a doctor. In an embodiment, the CT scan images may be saved on the database 114 and may be displayed on the I/O device 108 for generation of medical report. The user may be a radiologist and may view the CT scan images on the display screen and provide instructions via a voice input. In an embodiment, the database 114 or the memory 106 may include a predefined set of activation prompts. In an embodiment, each activation prompt when detected in the voice input of the user may enable the computing device 102 to perform a predefined activity or processing as discussed in detail below.

Based on detection of a first activation prompt in the voice input from the predefined set of activation prompts, the computing device 102 may determine a set of segmented images from each of the set of images. The set of segmented images may be determined by segmenting the body part, an anomaly in the body part and a background in each of the set of images using a segmentation model. The segmentation model may include, but not limited to, UNet architecture model. In an embodiment, the body part may be an internal organ such as liver, kidney, colon, etc. Further, the anomaly in the body part may be an unwanted growth such as tumours, polyps, or abscesses, etc. Further, the set of segmented images may be determined based on detection of the body part in the set of images and the anomaly in the body part and the background.

Further, the computing device 102 may determine a volumetric information of the anomaly in the body part from each of the set of segmented images. In an embodiment, the volumetric information may include volume information of the anomaly and area information of the anomaly determined in each of the segmented images. In an exemplary embodiment, the anomaly may include one or more tumours, wherein the volumetric information may include a number of tumours determined in each of the set of segmented images. Further, each of the one or more tumours in each of the set of segmented images may be determined based on detection of white pixels in the set of segmented images. Further, the volume information may be determined based on determination of a 3D volume of each of the one or more tumours. In an embodiment, the 3D volume of a corresponding tumour may be determined based on a voxel size of the white pixels representing the one or more tumours in each of the set of segmented images and a slice thickness of each of the set of segmented images. Further, the computing device 102 may output the set of segmented images and the volumetric information of the anomaly in the body part for each of the set of segmented images on the display device upon detection of the first activation prompt.

The computing device 102 may further determine text data from the voice input using a speech-to-text model based on detection of a second activation prompt from the pre-defined set of activation prompts in the voice input. In an embodiment, a second activation prompt may be input by the user based on analysis of the set of images, the set of segmented images and/or the volumetric information of the anomaly in the body part for each of the set of segmented images displayed on the display device of the I//O devices 108.

Further, the computing device 102 may generate the medical report based on the text data and the volumetric information. In an embodiment, the computing device 102 may output the medical report on the display device of the I//O devices 108 based on detection of a third activation prompt from the pre-defined set of activation prompts in the voice input. Further, the speech-to-text model may be disabled based on the detection of the third activation prompt.

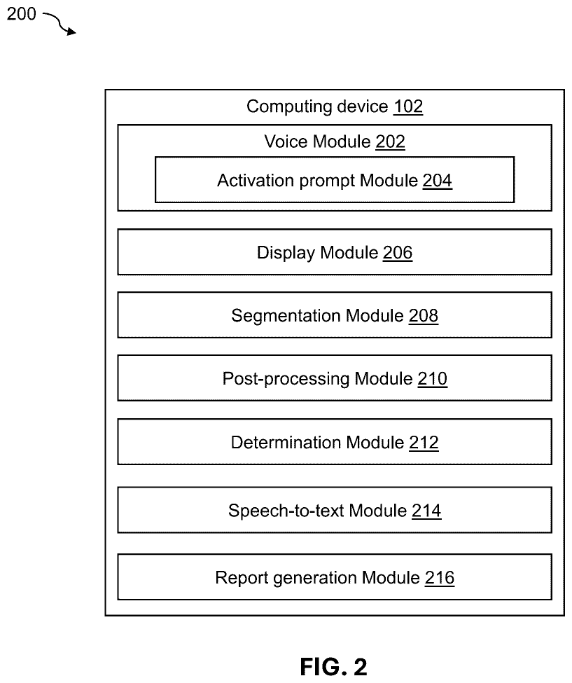

Referring now to FIG. 2, a functional block diagram 200 of the computing device 102 of the medical report generation system 100 of FIG. 1, in accordance with some embodiments of the present disclosure. In an embodiment, the computing device 102 may include a voice module 202, an activation prompt module 204, a display module 206, a segmentation module 208, a post-processing module 210, a determination module 212, a speech-to-text 214 and a report generation module 216.

The voice module 202 may receive a voice input from a user via the I/O device 108. Further, the voice module 202 may include an activation prompt module 204 may include a set of pre-defined activation prompts. The set of pre-defined activation prompts may act as a reference and based on detection of any of the pre-defined set of activation prompts in the voice input the computing device 102 may initialize a corresponding process for generation of medical report. It is to be noted each of the pre-defined set of activation prompts may be associated to a predefined process for generating the medical report. In an exemplary embodiment, the pre-defined set of activation prompts may include following activation prompts and the corresponding predefined processes for example “Start App”—to wake or execute the application, “Start inferencing”—to trigger the segmentation model and get volumetric data, “Start reporting”—to trigger speech-to-text model, “Stop reporting”—to stop speech-to-text model, and “End App”—to close the application”. Accordingly, the user may initiate or execute a report generation application by providing activation prompt “Start App” as voice input. Based on initiation of the report generation application, the computing device 102 may receive a set of images that may include a body part of a patient. The set of images may be CT scan images of the body part, and the body part may include one or more anomalies. In an embodiment, the set of images may be saved in the memory 106 or the database 114 in a Neuroimaging Informatics Technology Initiative (NIfTI) format. Further, the NIfTI format may include multiple slices of the CT scan images of the body part captured from different angels in order to capture the complete body part using medical equipment or scanning device. Examples of medical equipment or scanning device may may include but not limited to a CT scanner. Further, the display module 202 may display the set of images on a display device of the I/O devices 108. In an exemplary scenario, the user may view the set of images displayed on the display to analyse the body part of a patient for diagnosis.

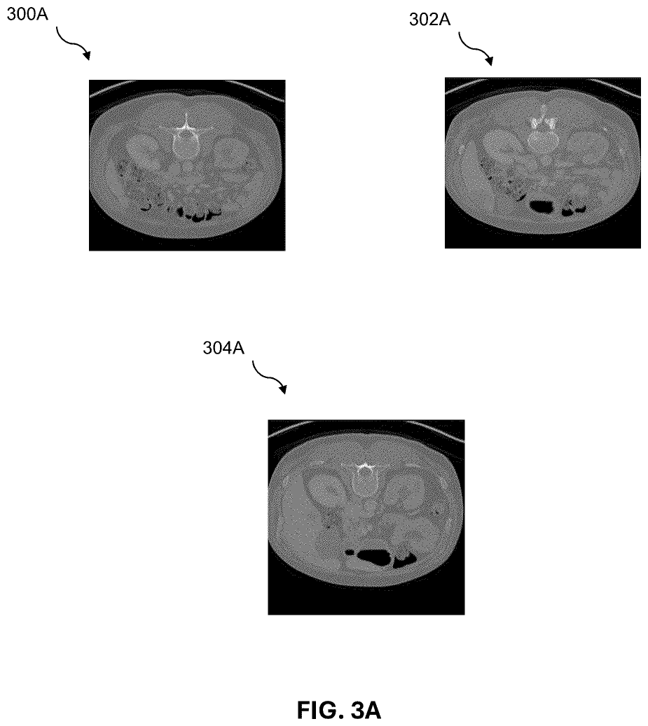

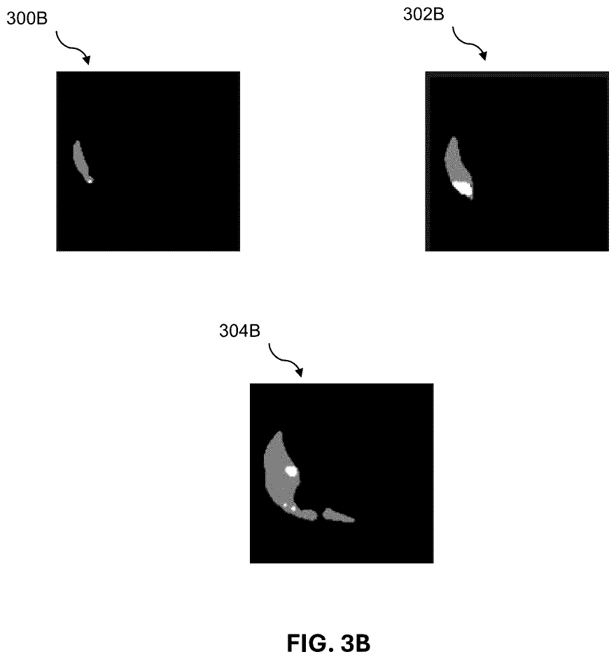

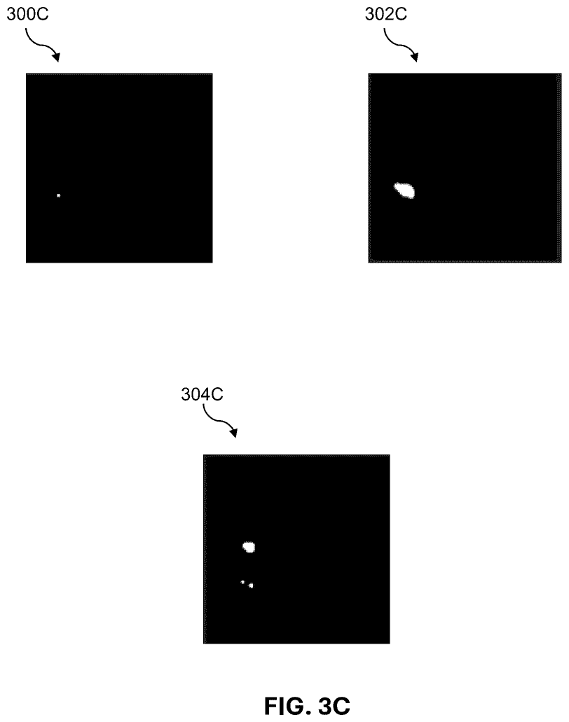

Referring to FIG. 3A, an exemplary set of images 300A, 302A and 304A of the body part are illustrated, in accordance with some embodiments of the present disclosure. The set of images 300A, 302A and 304A may be received by the display module 206 from an imaging device or the database 114. The set of images 300A, 302A, 304A are CT scans of liver as body part having tumour as anomaly. Further, the set of images 300A, 302A and 304A may be displayed by the display module 206.

Further, the user may continue to provide voice input and may initiate the segmentation module 208 by providing a first activation prompt from the pre-defined set of activation prompts by way of the voice input. In accordance with the exemplary embodiment, the first activation prompt may be “Start inferencing” based on detection of which the set of images 300A, 302A and 304A may be inputted to the segmentation module 208. The segmentation module 208 may determine the set of segmented images from each of the set of images 300A, 302A and 304A by segmenting the body part, the anomaly in the body part and the background using a segmentation model. In an embodiment, segmentation of the set of images may be performed based on the image's pixel characteristics, such as color, texture, or edge characteristics. In an embodiment, examples of segmentation model used may be, but not limited to, UNet segmentation model. In an embodiment, the segmentation model may be a UNet model that takes the CT scan images as input and may reduce resolution of the images while capturing important features and further highlight the important features like the body part, the anomaly, and the background. In an embodiment, the segmentation model may highlight the background using black pixels, the body part may be highlighted with grey pixels and the anomaly may be indicated using white pixels. In an embodiment, the anomaly may include one or more tumours that may be determined based on detection of white pixels in the set of segmented images.

FIG. 3B illustrates an exemplary set of segmented images 300B, 302B and 304B of the input images of FIG. 3A. the set of post segmented images 300B, 302B and 304B may be determined by the segmentation model of the segmentation module 208. As can be seen, the segmented images 300B, 302B and 304B depict the segmented body part i.e. liver in grey scale. The tumour detected in the liver as anomaly is depicted using white pixels and the background is depicted as black.

Further, the post processing module 210 may post-process the segmented images 300B, 302B, 304B in order to retain the anomaly information and by removing the body part. Referring to FIG. 3C, exemplary set of post-processed images 300C, 302C, 304C of the exemplary set of segmented images 300B, 302B and 304B of FIG. 3B are illustrated. The post-processing module 210 may process the segmented images 300B, 302B, 304B to remove the liver by removing the grey pixels and only retains the white pixels that may show the possible anomaly.

Further, the determination module 212 may determine the volumetric information of the anomaly in the body part in each of the set of segmented images 300B, 302B and 304B. In an embodiment, the volumetric information may include volume information of the anomaly and area information of the anomaly determined in each of the segmented images 300B, 302B and 304B. In an embodiment, the anomaly may include one or more tumours. In an embodiment, the one or more one or more tumours may be determined based on detection of white pixels in the set of segmented images 300B, 302B and 304B. In an embodiment, the area information of the anomaly may be determined by determining an area of the white pixels corresponding to each of the one or more tumours. Further, the volume information may be determined based on the 3D volume of each of the one or more tumour. The 3D volume of each of the one or more tumours may be calculated based on a voxel size of the white pixels in each of the set of segmented images 300B, 302B and 304B and the slice thickness of each of the segmented images 300B, 302B and 304B. In an embodiment, the set of images 300A, 302A and 304A and the volumetric information of the anomaly in the body part from each of the set of segmented images 300B, 302B and 304B may be outputted by the display module 206 upon detection of the first activation prompt.

Further, the speech-to-text module 214 may be activated based on detection of a second activation prompt from the pre-defined set of activation prompts in the voice input by the voice module 202. An example of the second activation prompt may include, but not limited to, “the Start reporting” that may trigger the speech-to-text module 214. In an embodiment, the second activation prompt from the voice input is detected based on analysis of the set of images 300A, 302A and 304A, the set of segmented images 300B, 302B and 304B and/or the volumetric information of the anomaly in the body part from each of the set of segmented images 300B, 302B and 304B displayed by the display module 206. Example of the speech-to-text model may include, but not limited to, a whisper automatic speech recognition (ASR) model that may transcribe a recited speech into text in real time. In an embodiment, the whisper ASR model is a transformer-based encoder-decoder architecture that may split the input speech into 30-second chunks, convert the chunks into log-Mel spectrogram and later pass into the encoder to encode the audio. The user may narrate the diagnosis as voice input that may be converted to text data by the speech-to-text module 214 and stored in the memory 106.

Further, the report generation module 216 may further generate a medical report based on the text data and the volumetric information. In an embodiment, the report generation module 216 may include a template medical report that may be updated with the volumetric data and the text data including the diagnosis of the user based on the analysis of the displayed set of images, the set of segmented images and the volumetric data. The report generation module 216 may output the medical report on the display device based on detection of a third activation prompt from the pre-defined set of activation prompts in the voice input. Example of the third activation prompt may be “Stop reporting” based on detection of which the speech-to-text module 214 may be disabled and the medical report generated by the report generation module 216 may be output by the display module 206. The generated medical report may be based on the user analyses of the set of images, set of segmented images and the volumetric information of the anomaly in the body part of the patient.

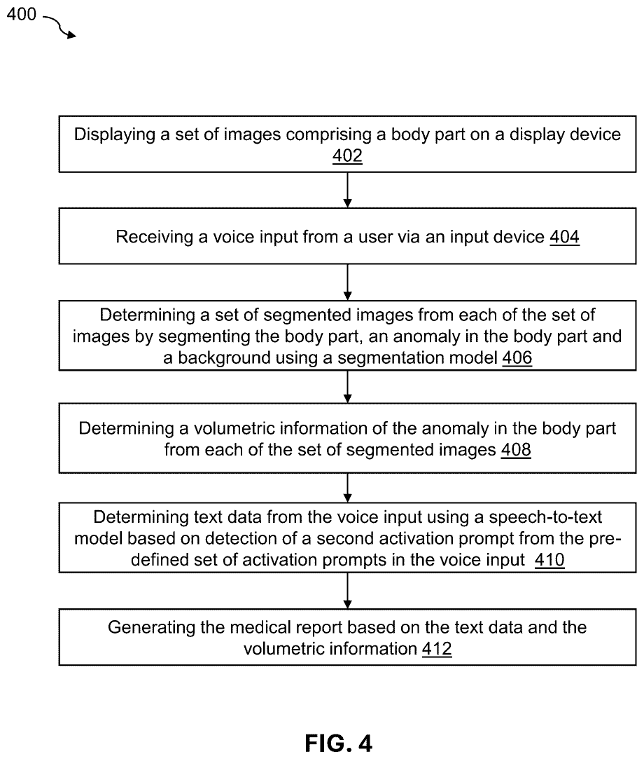

Referring to FIG. 4, a flow diagram 400 of a methodology of generating medical report, in accordance with some embodiments of the present disclosure. In an embodiment, the method 400 may include a plurality of steps that may be performed by the processor 104 to generate the medical report.

At step 402, the set of images 300A, 302A, 304A may be received by the computing device 102 and displayed by the display module 202 on a display device of the I/O devices 108. In an embodiment, the set of images 300A, 302A, 304A may be the CT scan images including a body part of the patient.

At step 404, the processor 104 may receive the voice input from the user. Now at step 406, the processor 104 may determine the set of segmented images 300B, 302B, 304B from each of the set of images 300A, 302A, 304A by segmenting the body part, the anomaly in the body part and the background using the segmentation model. In an embodiment, the set of segmented images 300B, 302B, 304B may be determined based on detection of a first activation prompt from a pre-defined set of activation prompts in the voice input received at step 404. Example of the first activation prompt may be “Start inferencing”—that may trigger the segmentation model and determination of volumetric data. The set of images 300A, 302A, 304A may be segmented based on detection of the body part that may be depicted with grey pixel. Further, the background may be depicted using black pixels and the anomaly present in the body part may be depicted using the white pixels.

Further at step 408, the processor 104 may determine the volumetric information of the anomaly in the body part from each of the set of segmented images 300B, 302B, 304B. In an embodiment, the volumetric information may include the volume information of the anomaly and area information of the anomaly determined in each of the segmented images 300B, 302B, 304B. In an embodiment, the anomaly may include one or more tumours, wherein the volumetric information comprises a number of tumours in each of the segmented images 300B, 302B and 304B. Further, each of the one or more tumours in each of the set of segmented images 300B, 302B, 304B may be determined based on detection of white pixels in the set of segmented images 300B, 302B, 304B. In an embodiment, the area information of the anomaly may be determined by determining an area of the white pixels corresponding to each of the one or more tumours. Further, the volume information may be determined based on determination of the 3D volume of each of the one or more tumours, wherein the 3D volume of a corresponding tumour may be determined based on the voxel size of the white pixels in each of the set of segmented images 300B, 302B, 304B and a slice thickness of each of the set of segmented images 300B, 302B, 304B. In order to determine the volume information, the set of segmented images 300B, 302B, 304B may be post-processed in order to retain the anomaly information by filtering out the area of the body part in the set of segmented images 300B, 302B, 304B. The post-processed images 300C, 302C, 304C may further be used to determine the volumetric information.

Further, the processor 104 may output the set of segmented images 300B, 302B, 304B and the volumetric information of the anomaly in the body part from each of the set of segmented images 300B, 302B, 304B on the display device upon detection of the first activation prompt.

At step 410, the processor 104 may determine the text data from the voice input using a speech-to-text model based on the detection of a second activation prompt from the pre-defined set of activation prompts in the voice input. In an embodiment, the second activation prompt from the voice input may be detected based on analysis of the set of images 300A, 302A, 304A, the set of segmented images 300B, 302B, 304B and/or the volumetric information of the anomaly in the body part from each of the set of segmented images 300B, 302B, 304B displayed on the display device. Further, the speech-to-text model may include but not limited to the ASR model that may take the voice input as speech data and transcribe it in real time to generate text data.

At step 412, the processor 104 may generate the medical report based on the text data and the volumetric information. In an embodiment, the text data may be determined by the speech-to-text model. In an embodiment, the medical report may be generated based on customization of a predefined template medical report.

It is intended that the disclosure and examples be considered as exemplary only, with a true scope of disclosed embodiments being indicated by the following claims.

What is claimed is:

1. A method of generating a medical report, the method comprising:
displaying, by a processor, a set of images comprising a body part on a display device;
receiving, by the processor, a voice input from a user via an input device;
determining, by the processor, a set of segmented images from each of the set of images by segmenting the body part, an anomaly in the body part and a background using a segmentation model,
wherein the set of segmented images are determined based on detection of a first activation prompt from a pre-defined set of activation prompts in the voice input;
determining, by the processor, a volumetric information of the anomaly in the body part from each of the set of segmented images,
wherein the volumetric information comprises volume information of the anomaly and area information of the anomaly determined for each of the segmented images;
determining, by the processor, text data from the voice input using a speech-to-text model based on detection of a second activation prompt from the pre-defined set of activation prompts in the voice input; and
generating, by the processor, the medical report based on the text data and the volumetric information.

2. The method of claim 1, comprising outputting, by the processor, the set of segmented images and the volumetric information of the anomaly in the body part for each of the set of segmented images on the display device upon detection of the first activation prompt.

3. The method of claim 2, wherein the second activation prompt from the voice input is detected based on analysis of the set of images, the set of segmented images and/or the volumetric information of the anomaly in the body part from each of the set of segmented images displayed on the display device.

4. The method of claim 1, comprising:
outputting, by the processor, the medical report on the display device based on detection of a third activation prompt from the pre-defined set of activation prompts in the voice input; and
disabling, by the processor, the speech-to-text model upon the detection of the third activation prompt.

5. The method of claim 1, wherein the anomaly comprises one or more tumours, wherein the volumetric information comprises a number of tumours in each of the set of segmented images.

6. The method of claim 5, wherein the one or more tumours in each of the set of segmented images are determined based on detection of white pixels in the set of segmented images.

7. The method of 6, wherein the area information of the anomaly is determined by determining an area of the white pixels corresponding to each of the one or more tumours.

8. The method of claim 7, wherein volume information is determined based on determination of a 3D volume of each of the one or more tumours, wherein the 3D volume of a corresponding tumour is determined based on a voxel size of the white pixels in each of the set of segmented images and a slice thickness of each of the set of segmented images.

9. A system for generating a medical report, comprising:
a processor; and
a memory communicably coupled to the processor, wherein the memory stores processor-executable instruction, which, on execution by the processor cause the processor to:
display a set of images comprising a body part on a display device;
receive a voice input from a user via an input device;
determine a set of segmented images from each of the set of images by segmenting the body part, an anomaly in the body and a background using a segmentation model,
wherein the set of segmented images are determined based on detection of a first activation prompt from a pre-defined set of activation prompts in the voice input;
determine a volumetric information of the anomaly in the body part from each of the set of segmented images,
wherein the volumetric information comprise volume information of the anomaly and area information of the anomaly determined in each of the set of segmented images;
determine text data from the voice input using a speech-to-text model based on detection of a second activation prompt from the pre-defined set of activation prompts in the voice input; and
generate the medical report based on the text data and the volumetric information.

10. The system of claim 9, wherein the processor is configured to:
output the set of segmented images and the volumetric information of the anomaly in the body part from each of the set of segmented images on the display device upon detection of the first activation prompt.

11. The system of claim 10, wherein the second activation prompt from the voice input is detected based on analysis of the set of images, the set of segmented images and/or the volumetric information of the anomaly in the body part from each of the set of segmented images displayed on the display device.

12. The system of claim 9, wherein the processor is configured to:
output the medical report on the display device based on detection of a third activation prompt from the pre-defined set of activation prompts in the voice input; and
disable the speech-to-text model upon the detection of the third activation prompt.

13. The system of claim 9, wherein the anomaly comprises one or more tumours, wherein the volumetric information comprises a number of tumours in each of the set of segmented images.

14. The system of claim 13, wherein the one or more tumours in each of the set of segmented images are determined based on detection of white pixels in the set of segmented images.

15. The system of claim 14, wherein the area information of the anomaly is determined by determining an area of the white pixels corresponding to each of the one or more tumours.

16. The system of claim 15, wherein volume information is determined based on determination of a 3D volume of each of the one or more tumours, wherein the 3D volume of a corresponding tumour is determined based on a voxel size of the white pixels in each of the set of segmented images and a slice thickness of each of the set of segmented images.

17. A non-transitory computer-readable medium storing computer-executable instructions for generating a medical report, the computer-executable instructions configured for:
displaying a set of images comprising a body part on a display device;
receiving a voice input from a user via an input device;
determining a set of segmented images from each of the set of images by segmenting the body part, an anomaly in the body part and a background using a segmentation model,
wherein the set of segmented images are determined based on detection of a first activation prompt from a pre-defined set of activation prompts in the voice input;
determining a volumetric information of the anomaly in the body part from each of the set of segmented images,
wherein the volumetric information comprises volume information of the anomaly and area information of the anomaly determined for each of the segmented images;
determining text data from the voice input using a speech-to-text model based on detection of a second activation prompt from the pre-defined set of activation prompts in the voice input; and
generating the medical report based on the text data and the volumetric information.

18. The non-transitory computer-readable medium of claim 17, wherein the computer-executable instructions are configured for:
outputting the set of segmented images and the volumetric information of the anomaly in the body part for each of the set of segmented images on the display device upon detection of the first activation prompt

19. The non-transitory computer-readable medium of claim 18, wherein the second activation prompt from the voice input is detected based on analysis of the set of images, the set of segmented images and/or the volumetric information of the anomaly in the body part from each of the set of segmented images displayed on the display device.

20. The non-transitory computer-readable medium of claim 17, wherein the computer-executable instructions are configured for:
outputting the medical report on the display device based on detection of a third activation prompt from the pre-defined set of activation prompts in the voice input; and
disabling the speech-to-text model upon the detection of the third activation prompt.
